(19) United States
(12) Patent Application Publication
(10) Pub. No.: US 2018/0160977 A1
(43) Pub. Date: Jun. 14, 2018

(54) PORTABLE JOINT TESTING DEVICE

(71) Applicants: Patrick A. Meere, Yorktown Heights, NY (US); Matthias André Verstraete, Heerweg-Zuid (BE)

(72) Inventors: Patrick A. Meere, Yorktown Heights, NY (US); Matthias André Verstraete, Heerweg-Zuid (BE)

(21) Appl. No.: 15/836,705

(22) Filed: Dec. 8, 2017

Related U.S. Application Data
(60) Provisional application No. 62/432,440, filed on Dec. 9, 2016.

(51) Int. Cl. A61B 5/00; A61B 5/11

(52) U.S. Cl. CPC A61B 5/6828; A61B 5/4576; A61F 2002/4666; A61B 5/4585; A61B 5/1107; A61B 5/1121

(57) ABSTRACT

A portable joint testing device that generates reproducible measurements of rotation about all three axes with load-bearing activities includes a first position sensor mounted on a first bone proximate the joint for transmitting first position data; a second position sensor mounted on the second bone proximate the first joint for transmitting second position data; and at least one force sensor mounted on the second bone for quantifying forces applied to the second bone and for transmitting force data. A controller collects the force data and the first and second position data, and transmits or generates a laxity envelope for varus/valgus and/or internal/external rotation of the first bone relative to the second bone or vice versa.

### CROSS REFERENCE TO RELATED APPLICATIONS

This application claims priority from U.S. Patent Application No. 62/432,440, filed Dec. 9, 2016, which is incorporated herein by reference.

### TECHNICAL FIELD

The present invention relates to a portable joint testing device, and in particular to a portable joint testing device for pre and post-operative testing of the range of motion and laxity of a joint.

### BACKGROUND

The major joints in the human body are classified as diarthrosis joints. These joints link major limb segments and can be further classified based on their structure and function. Most have a low friction surface, such as cartilage, with a closed capsular envelope, lined by a synovial lining layer, which produces lubrication fluid. The motion of the joint is determined by the matching morphology of the opposing surfaces. The stability is critically dependent on passive structures, such as capsule and ligaments, and coordinated active muscle control. When injured by trauma or mechanical overuse, or when damaged by aging or disease, the joint loses flexibility and strength and may become highly unstable, compounding wear through asymmetrical shear forces. Surgery and extensive rehabilitation intend to diminish pain and restore function. The success depends on restoration of a harmonious balance between active and passive stabilizing structures of the joint. The relative efficiency of treatment can be judged on patient satisfaction and function scores (Patient Reported Outcome Measures). There is however growing dissatisfaction because of the subjective nature of these scores. Increasingly third party payers rely on the delta value of standardized tests before and after surgery. The problem is that the classical test for ligamentous laxity of the knee (for example) is considered by the American Medical Association (AMA) as inefficient and inaccurate. There is no control of input forces and no quantitative assessment of soft tissue envelope. Furthermore, the tests suffer from high variance in their execution and are performed with the patient non-weightbearing.

The advent of sensor technology now allows for precise assessment of the compartmental load distribution at the time of surgery. This combined with precise execution of the bone alignment and bone cuts, known as measured resection, has the potential to raise the patient satisfaction, function and long term results. These advanced surgical techniques suffer from low adoption rates because of the lack of demonstrable objective proof of superiority (subjectivity of patient self-reporting).

The goal of a portable, reliable and precise joint sleeve-brace equipped with sensors is to enable systematic measurement and recording of the knee motion performance through the episode of care from injury or surgery through completion of rehabilitation. The current available technology is bulky, non-portable and cannot measure live forces of dynamic loaded motion. Examples of ligamentous testing of long bone joints include such devices as the KT-1000™, and Telos™ device for antero-posterior displacement and rotational laxity in the frontal plane respectively. These devices are large vise-like machines that deliver three point forces when the patient's limb is fitted into them. They have no claims to assist in the field or the operating theater. Therapeutic braces are numerous and serve as mechanical restraints or passive-assisted guides to help with stabilization of the joint. They are not a long term solution and are often frowned upon as they inhibit the ability to develop the necessary muscle strength to guide joint motion without external help.

Sensor enabled or instrumented braces have been developed in the last decade to measure specific functional parameters. Unfortunately, these attempts are not suitable for a surgical environment where sterile access to the tissues of the joint is required. In addition, measuring only a single degree of freedom does not provide feedback of the joint stability through the range of motion. The assessment of clinical stability remains thus very subjective and imprecise.

An object of the present invention is to overcome the shortcomings of the prior art by providing a portable joint testing device that provides reproducible measurements of rotation about all three axes with load-bearing activities and standard activities of daily living.

### SUMMARY OF THE INVENTION

Accordingly, the present invention relates to a testing device to determine degrees of freedom of a first joint, defined by first and second bones, comprising:

at least one first position sensor capable of being mounted on the first bone proximate the first joint for transmitting first position data;

at least one second position sensor capable of being mounted on the second bone proximate the first joint for transmitting second position data;

at least one force sensor capable of being connected to the second bone for quantifying forces applied to the second bone and for transmitting force data;

a controller for collecting the force data and the first and second position data; and

a force applicator mounted on the end of the second bone capable of transmitting an applied force to the end of the second bone.

Another aspect of the present invention relates to a method of evaluating the stability of a first joint comprising:

attaching angular position sensors respectively to a first and a second bone above and below the first joint;

attaching at least one force sensor proximate an outer free end of the second bone,

mounting a force applicator proximate an outer free end of the second bone;

applying varus/valgus forces and internal/external rotational torques to the second bone while moving through the flexion-extension range of motion with the first joint;

generating angular motion data with the angular position sensors;

generating force data with the at least one force sensor; and

using a central processing unit in communication with the angular position sensors and the force sensors, for calculating angular positions of the first joint in terms of varus/valgus rotation, flexion/extension and internal/external rotation.

Another feature of the present invention provides a method to use, transfer and apply clinically obtained multi-dimensional laxity data in the field during activities of daily living, rehabilitation and sports activities, whereby the data includes:

in a clinical environment, identifying joint limits comprising angular positions that require elevated or steeply increasing levels of applied forces;

in a non-clinical environment, identifying a set of multi-dimensional joint positions through the range of motion of the joint in relation to external forces required to reach the positions; and

providing direct feedback to the user when said established joint limits are approached or exceeded for a given degree of freedom, where said limits depends on at least one of:

an angular position of the joint;

an angular position of the joint as a function of another angular degree of freedom; and

an angular position of the joint as a function of the clinically required force to reach that position.

### BRIEF DESCRIPTION OF THE DRAWINGS

The invention will be described in greater detail with reference to the accompanying drawings which represent preferred embodiments thereof, wherein:

FIG. 1 is a front view of an embodiment of the present invention;

FIG. 2 is an isometric view of another embodiment of the present invention;

FIG. 3a is a bottom view of a force applicator of the device of FIG. 2;

FIG. 3b is a cross sectional view of the force applicator of FIG. 3a;

FIG. 4 is a schematic view of an embodiment of the system of the present invention;

FIG. 5 is a schematic view of an embodiment of the system of the present invention;

FIG. 6 is a schematic view of an embodiment of the system of the present invention; and

FIG. 7 is a schematic view of an embodiment of the system of the present invention.

### DETAILED DESCRIPTION

While the present teachings are described in conjunction with various embodiments and examples, it is not intended that the present teachings be limited to such embodiments. On the contrary, the present teachings encompass various alternatives and equivalents, as will be appreciated by those of skill in the art.

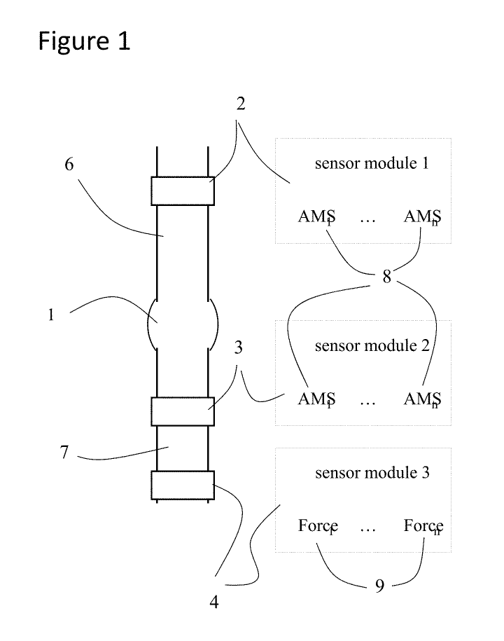

An embodiment of the present invention, illustrated in FIG. 1, evaluates a range of motion of a joint 1, e.g. knee, elbow or shoulder, for all angular degrees of freedom by combining the readings from at least three different sensor modules 2, 3 and 4, that are placed at the body segments 6 and 7 adjacent to and on either side of the joint 1, e.g. first limb 6 (femur) and second limb 7 (tibia). In the illustrated embodiment, three different, synchronized sensor modules may be included in this invention: a first and a second angular position modules 2 and 3, respectively, and a first force module 4.

The first and second angular position modules 2 and 3 may come in at least two different versions: a first version comprising sensors that are integrated in a stretchable substrate fitted around the user's limbs, and a version whereby the sensors are placed in an external box that may be attached/detached to an elastic band. Both the elastic substrate and stretch band have a tight fit with the underlying body segments 6 and 7 aiming to minimize their relative movement. For both versions, each of the angular position modules 2 and 3 may comprise one or more angular measurement sensors (AMS) 8₁-8ₙ. A plurality of different sensors 8₁-8ₙ, e.g. 3 or more spaced apart longitudinally along the length of the body segment 6 or 7 and/or laterally around the body segment 6 and 7, may be combined to minimize the detrimental effects caused by the movement of the underlying soft tissues, whereby dedicated algorithms exist that filter the measured signals and eliminate anomalous values and/or average between the different AMS's 8₁-8ₙ. Regarding the first version of the angular position module 2 and 3, the stretching substrate may be seen either as a functional piece of clothing made of elastic material that spans multiple body segments or as a band of elastic material that only seats the investigated body segment 6 or 7. The AMS's 8₁-8ₙ are consequently integrated in the elastic material and may be interconnected through wires or wireless transmitters that are integrated in the elastic material.

The first force module 4 measures the forces that are exerted on the joint 1, e.g. knee, elbow or shoulder during the testing. The first force module 4 may be connected to one of the body segments 6 or 7 on either side of the studied joint 1 and held by the evaluator. The connection is either direct, by attaching the first force module 4 to the body segment 6 or 7 adjacent to the joint 1, or indirect by attaching the first force module 4 to a distal body segment 8, e.g. foot or hand, further away from the joint 1, and locking any intermediate joints, e.g. ankle or wrist. The evaluator thereby moves the joint 1 through the range of motion by holding the first force module 4. The force exerted by the evaluator is subsequently recorded by the first force module 4, while the AMS's 8₁-8ₙ record the angular positions or the body segments 6 and 7 and the joint 1. The first force module 4 may comprise a plurality of different force sensors 9₁-9ₙ that are used to quantify the exerted forces in individual, potentially unrelated, directions. The force sensors 9₁-9ₙ may be place longitudinally along the length of the body segment 6 or 7, and laterally around the circumference.

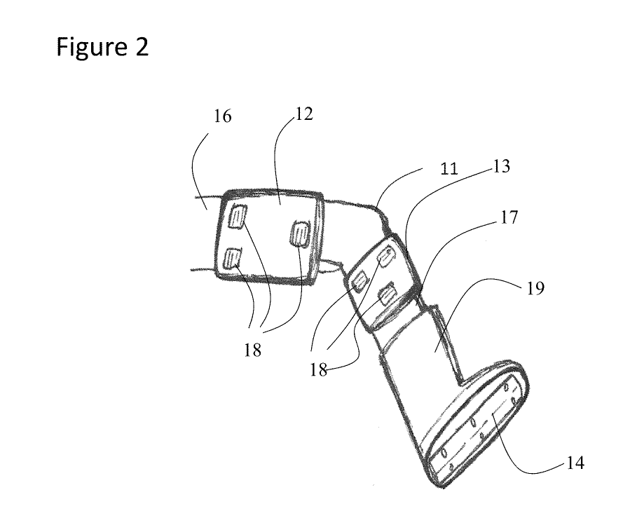

With reference to FIG. 2, an example application of the present invention involves the evaluation of the laxity and range of motion of a knee joint 11. First and second angular position modules 12 and 13 are mounted on either side and proximate to the knee joint 11, e.g. on the lower femur 16 and upper tibia 17, respectively. Both options for the first and second angular measurement modules 12 and 13 may rely on the use of one or more inertial measurement units (IMU) 18₁-18ₙ. The IMU sensors 18₁-18ₙ may be integrated in a stretching pant, trouser or femoral and tibial band. Alternatively, the IMU sensors 18₁-18ₙ may be integrated in a rigid box that may be attached/detached to an elastic band that fits around the femur 16 or tibia 17.

To evaluate the externally applied forces, the load sensors 33₁ to 33ₙ may be integrated in a rigid boot 19 (or glove or elbow brace) that seats on one or more adjacent body segments, e.g. the distal tibia 17 and foot (radius or humerus). The boot 19 immobilizes an adjacent joint, e.g. the ankle joint, such that forces exerted at the foot (or hand) are transferred to the tibia 17 without major deflection of the ankle joint, as to preserve the orientation of the applied forces with respect to the tibia 17. The boot 19 may be replaced by a suitably fitting glove for immobilizing the wrist during elbow examinations or an elbow brace for immobilizing the elbow during shoulder testing. At the bottom of the boot 19, a multi-axial force sensor 14 may be attached.

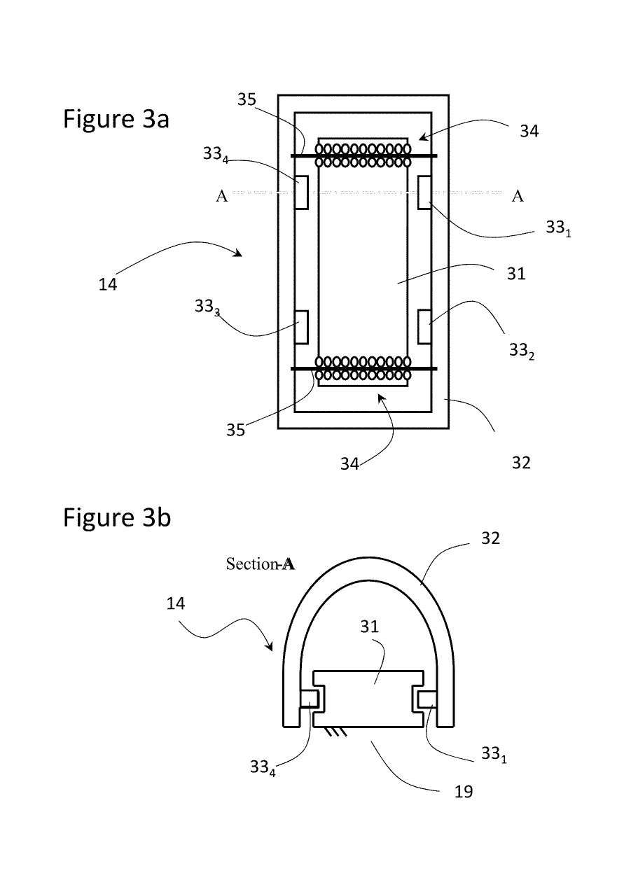

With reference to FIGS. 3a and 3b, the force sensor module 14 may be a hand-held unit that comprises a central plate 31, an outside cover 32, and load sensors 33₁ to 33ₙ. The central plate 31 may thereby be coupled to the boot 19, either permanently or through a magnetic click-on/shape fitted snap-on connection. The cover 32 may be shaped in such a way that it includes a handle for fitting a hand of the evaluator. Ideally, the connection between the cover 32 and the plate 31 is such that the load sensors 33₁ to 33ₙ record uni-axial forces. Therefore, a combination of load sensors 33₁ to 33ₙ and force guides 34 may be used. In an example, the force guides 34 may comprise guided rods 35, which allow free motion along their longitudinal direction, but transfer all loads in the perpendicular directions, i.e. perpendicular to longitudinal axis of the limb 17 or perpendicular to the plane described by the longitudinal/mechanical axis of the tibia and the anteroposterior axis of the foot. The associated load sensors 33₁ to 33ₙ capture the applied forces in the longitudinal direction of the guided rods 35. As a result, the load sensors 33₁ to 33ₙ are shielded from shear forces. In the illustrated example, four distinct spaced apart load sensors 33₁ to 33ₙ are integrated in the force sensor 14, two on each side of the plate 31. Each load sensor 33₁ to 33ₙ comprises a uni-axial, uni-directional sensor that captures the exerted compressive load on the plate 31 and therefore the joint 11. From the readings of load sensors 33₁ to 33ₙ, the externally applied varus-valgus load and the applied internal-external rotational torque on the joint 11 may be calculated.

The abovementioned combination of load sensors 33₁ to 33ₙ and angular position sensors 8 or 18 come in different varieties. A first alternative combines the tibial motion sensors 8 or 18 and load sensors 33₁ to 33ₙ in a single device, e.g. the boot, glove or elbow brace 19. Combining the load and motion sensors in a single device; however, bears the risk that soft tissue movement through the application of loads results in anomalous, exaggerated angular position movements of the body segment 17. In a second alternative topography, the presented load sensors 33₁to 33ₙ are somewhat miniaturized and integrated in the sole of the boot, glove or elbow brace 19, and are not detachable and useable for continuous monitoring. Third, some of the load sensors 33₁ to 33ₙ may be located around the medial and lateral malleoli, such that the varus/valgus load should not be applied at the sole of the boot 19, but rather at the medial and lateral distal end of the tibia 17 (radius or humerus).

To give a meaningful interpretation to the signals that originate from the angular position modules 2/12, 3/13, a dedicated calibration algorithm may be integrated. The calibration algorithm aims to identify the coronal, sagittal and axial plane for all involved segments, e.g. bones 6/16, 7/17 and the joint 1/11. The calibrations method may be self-administered by the patient or, alternatively, performed by a third party under controlled conditions, to enhance the accuracy and reproducibility of the measurements. The calibration process may combine or rely on different algorithms, three of which are described hereafter. The first calibration process includes a series of swing movements of the bones 6/7 or 16/17 and joint 1/11 in question in dedicated, isolated anatomical planes, and as such identify the orientation of the sensors 8 or 18 with respect to their respective body segments. A second possible calibration process includes moving through the range of motion of the limbs 6/7 or 16/17, and as such evaluate the joint coordinate system through the identification of the dominant movement direction. A third calibration process includes keeping the joint 1, 11 at predefined fixed positions during a limited period of time.

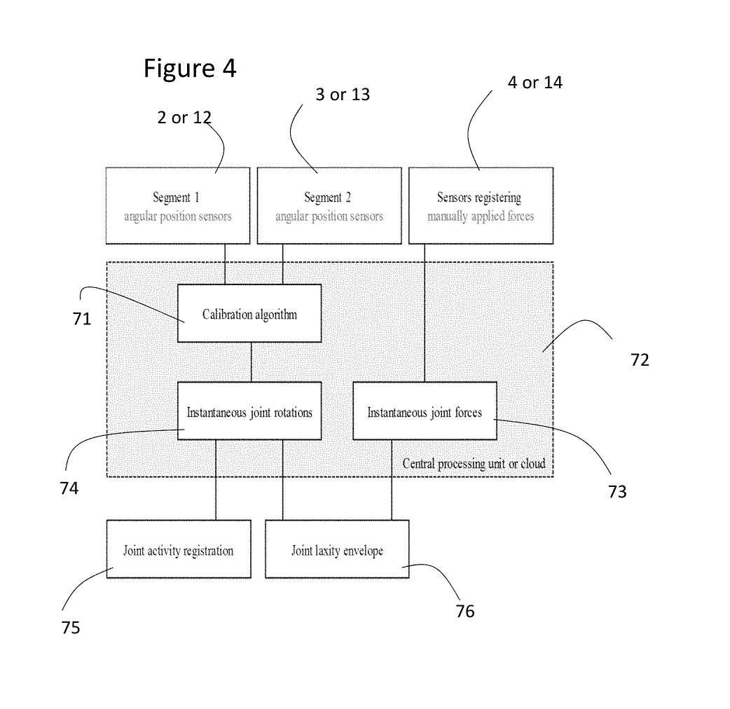

With reference to FIG. 4, following the installation of the sensor modules 2/12, 3/13, 4/14, and calibration 71 of the sensors 8₁-8ₙ or 18₁-18ₙ and 9₁-9ₙ or 33₁ to 33ₙ, the signals from the sensors are transferred, e.g. with wire or wirelessly, to a central processing unit 72 that has the software saved in non-transitory memory and executable on the CPU to combine and interpret the sensor readings. More specifically, the applied forces 73 as well as the recorded angular positions 74 are transferred to the joint coordinate system facilitating a physical and potentially meaningful interpretation of the measurement data to provide a joint activity registration 75 and a joint laxity envelope 76 for each joint 1 or 11 tested. The exact calculations thereby differ from the application.

At least two distinct applications are provided by the present invention. First, the individual sensor readings from the angular position sensors 8₁-8ₙ or 18₁-18ₙ may be collected over a longer period of time, e.g. days, week, months or years, and combined to provide insight in the activity, e.g. number of cycles, range of cycles, frequently performed ranges, and multidimensional range of motion of a particular joint 1 or 11. The measurements may be performed in the clinic, but also during activities of daily life outside a clinical setting, using so-called wearable ROM sensors by integrating the sensors in a set of stretchable sleeves, e.g. trousers. In the latter case, the interpretation and registration of the measurements potentially contributes to the rehabilitation process or identification and diagnosis by objectifying the patient complaints.

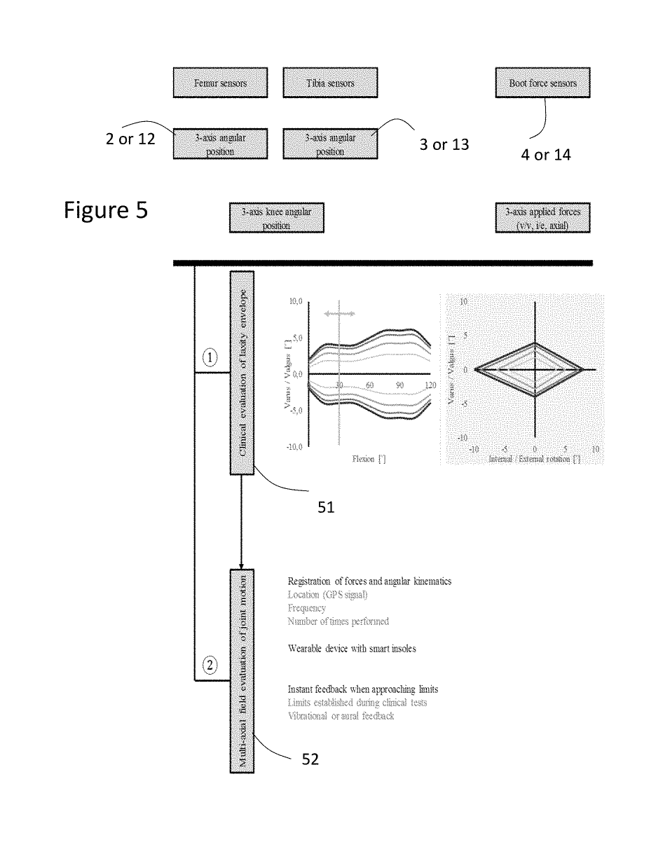

With reference to FIG. 5, a first application of the present invention comprises a personal monitoring/warning system for an individual wishing to monitor a specific joint, e.g. 1 or 11. An initial step comprises a clinical evaluation 51 of the multi-dimensional laxity envelope of a given joint 1 or 11. The evaluation may be performed by an evaluator by applying a set of predefined loads to the joint 1 or 11, e.g. knee, throughout the range of motion, as further described in the second application below. The readings from the different sensor 2/3/4 or 12/13/14 may be first combined in a clinical device that is used for a quantitative analysis of the joint laxity by logging the forces applied by the clinician synchronously to the joint angles. The resulting multi-dimensional laxity envelope describes the varus-valgus and internal-external rotational laxity throughout the range of motion of the joint 1 or 11. (See plots in FIG. 5) Second, the data from the clinical testing is loaded into a field device, e.g. a wearable or portable ROM sensor such as a smart watch or smart phone, for the individual, which contains dedicated algorithms to identify the multi-dimensional angular limits from the test data obtained in the controlled, clinical setting.

The field device comprises of 3-axis angular sensors 2/3/4 or 12/13/14 on the bones on either side of the joint 1 or 11, e.g. femur and tibia, to continuously evaluate the joint position, thereby obtaining a field evaluation 52. The current joint position is thereby continuously compared to the reference limits obtained from the clinical evaluation. The user thereby receives instant feedback when approaching or exceeding the clinically established limits. In addition, the multidimensional joint position is stored and made available for more extensive post-processing and feedback to the clinicians, rehab specialists or sports trainers. Special clothing may be obtained for non-clinical testing, including resilient sleeves, leggings, or pants, with the angular sensors 2/3 or 12/13 embedded therein. The force sensors 4 or 14 may be provided as a glove or footwear insert or ankle or wrist strap for measuring external forces impacting the second bone.

These algorithms may be based on any one or more of: the magnitude of the applied force, e.g. exceeding certain limits, the derivative of the applied force as function of the associated angular deformation, e.g. large force increase needed for marginal increase in angular position change, or simply the range of motion included in the test data, e.g. only tested to certain flexion angle. Based on these limits, the individual patient, wearing sensor modules 2/3/4 or 12/13/14 during activity, receives real-time feedback on his/her current angular joint position with respect to the in-clinic established limits, e.g. within the laxity envelope. For each flexion angle of the joint, these limits represent the varus/valgus and/or internal/external rotation corresponding to a critical relative increase in the required force per degree of movement for the considered angular degree of freedom. The patient thus receives relevant real-time guidance with respect to the performed activities from clinically examined parameters. Doing so, the patient identifies risk limits in real-life circumstances that might not be sensed otherwise. In turn, this has the potential to reduce the risks for sprain, inflammation or chronic over-use injuries. To facilitate an effective feedback mechanism, the wearable central processing unit 72 comprises a vibrational element or a tone producing element. For both elements, the magnitude and/or frequency are linked to the vicinity of the current position to the established limits of the joint in question. The patient may also download a software application onto their smart phone, smart watch, mobile device or even home computer, which either automatically receives the force/displacement information, if in direct communication with sensors 2/3/4 or 12/13/14, or may receive the force/displacement data along with time information, whenever the sensors 2/3/4 or 12/13/14 are linked thereto. The App may also communicate directly with the doctor's office over another suitable communications network, e.g. cellular or internet, to provide updated data in real time or whenever downloaded.

For a second application, the angular position measurements and the associated joint coordinates are combined with the external, manually applied loads. Subsequently, the angular deflections may be evaluated as function of the applied loads, defining the boundaries of laxity for the examined joint 1 or 11. This evaluation is potentially performed in different degrees of freedom using a portable, mobile device, e.g. sensor modules 2/3/4 or 12/13/14. This evaluation may be performed at different moments during the treatment of a pathological situation. A first evaluation is performed during the identification of the pathology by examining the deficient joint 1 or 11 and comparing the measurements to the contralateral side or a reference case obtained from a wider population or analog data obtained by comparing to other joints. Second, the evaluation is performed during surgery to assess the effectiveness of the treatment and need for further surgical modifications or interventions. Third, the evaluation may be performed at various times during rehabilitation to follow up on the rehab process and establish safe limits for the rehabilitation exercises. In addition to a pathological setting, the laxity limits and the method described in the current invention can also be used to train athletes or recreative sportsmen and women in the identification and quantification of their limits and for the prevention of injuries.

A more concrete application of the above, is presented by the evaluation of knee laxity and stability before, during and after surgery. Thereby, the boot 19 and a femoral strap sensor module 12 are first positioned followed by a calibration procedure 71. Subsequently, the involved medical professional may move the joint 1 or 11 through the range of motion, while applying a varying force and torque, e.g. to the handheld cover 32 at the bottom of the boot 19. As such, the joint 1 or 11 is subject to various varus/valgus and internal/external rotational torques. By representing the varus/valgus deflection or internal/external rotation subsequently as a function of the flexion angle and the magnitude of the applied load, the neutral path and boundaries of motion are visualized for the specific case. This representation and testing sequence results in a standardized and quantified assessment of the joint 1 or 11 that enables a comparison and an evaluation of the performance of the joint over time.

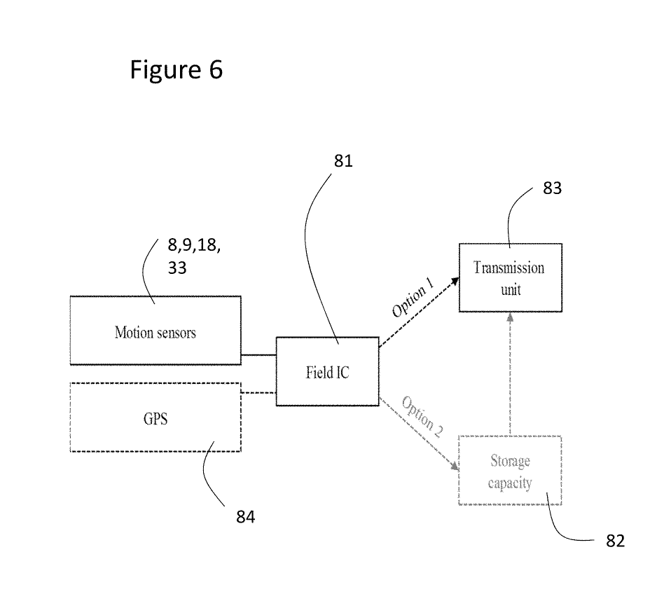

With reference to FIG. 6, the communication between the angular motion sensors 8 or 18, the load sensors 9 or 33₁ to 33ₙ and the central processing unit 72 is thereby performed either through a wired or wireless connection. Therefore, the actual angular sensors 8 or 18 and the load sensors 9 or 33₁ to 33ₙ are immediately connected to an integrated circuit 81 that has the potential to either locally store in integrated memory 82 (option 2) or transmit (option 1) the data via a transmission unit 83. The former is particularly relevant when the (motion) sensors 8 or 18 are used as a field capture device that provides direct feedback in addition to an overview following the performed activities. In that respect, the sensors 8 or 18 may be complemented by a GPS and/or time sensor 84 that enables the identification of the geographic location and position at which particular movements/instabilities were performed or experienced to enable correlation therebetween.

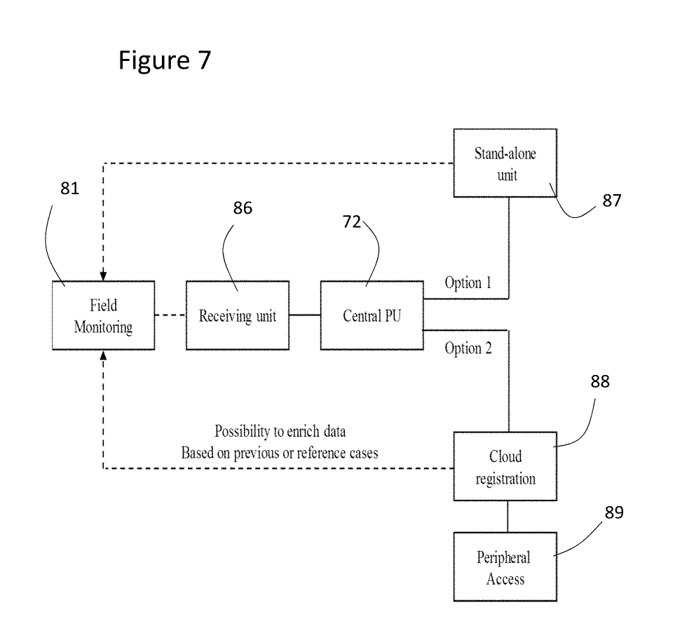

With reference to FIG. 7, the transmitted signal may subsequently be captured by a receiving unit 86 the central processing unit 72 that, in turn, has the ability to store the data in non-transitory memory, process the data using software stored in the non-transitory memory and executable by the CPU 72, and/or send feedback to the user/clinical examiner. In a first option, the receiving device acts as a stand-alone unit 87. In an alternative version, the data receiving device is connected to the internet and sends the data to a remote, e.g. cloud, database 88. By doing so, the recorded data may instantly be used to perform additional analyses and interpretation before sending feedback to the user. The feedback thereby includes the measured data, potentially complemented by historic and/or reference data for the particular joint and/or individual. The device that is thereby used to present the feedback is not necessarily the same as the receiving unit. As such, the receiving data can include a general purpose mobile device (e.g. mobile phone or tablet). Through the remote, e.g. cloud, storage, the sensor feedback may additionally be accessed by or sent to third parties 89, such as remote rehab trainers or medical professionals.

The combination of these sensors thus includes two different applications: a sports and rehab related application and a clinical application. The combination of both is crucial to solve the issues of overtraining and the establishment of safe training limits during rehab and regular/competitive sports applications.

The foregoing description of one or more embodiments of the invention has been presented for the purposes of illustration and description. It is not intended to be exhaustive or to limit the invention to the precise form disclosed. Many modifications and variations are possible in light of the above teaching. It is intended that the scope of the invention be limited not by this detailed description, but rather by the claims appended hereto.

We claim:

1. A testing device to determine degrees of freedom of a first joint, defined by first and second bones, comprising:
at least one first position sensor capable of being mounted on the first bone proximate the first joint for transmitting first position data;
at least one second position sensor capable of being mounted on the second bone proximate the first joint for transmitting second position data;
at least one force sensor capable of being connected to the second bone for quantifying forces applied to the second bone and for transmitting force data;
a controller for collecting the force data and the first and second position data; and
a force applicator mounted on the end of the second bone capable of transmitting an applied force to the end of the second bone.

2. The testing device according to claim 1, wherein the force applicator includes guides enabling free motion in a force application direction, while preventing motion in directions other than the force application direction.

3. The testing device according to claim 1, wherein the force applicator comprises a rigid material to minimize soft tissue movement during force application.

4. The testing device according to claim 1, wherein the force applicator includes a joint immobilizer for immobilizing a second joint between the second bone and a third bone connected to an end of the second bone, thereby transferring any of the applied force to the first joint via the second joint.

5. The testing device according to claim 1, wherein the force applicator comprises:
a joint immobilizer mounted on the end of the second bone over a second joint between the second bone and a third bone;
a frame for supporting the at least one force sensor proximate the joint immobilizer, reciprocateable relative to the joint immobilizer; and
guides for ensuring uniaxial movement of the frame relative to the joint immobilizer.
a central processing unit capable of evaluating the force data and the first and second position data.

6. The testing device according to claim 5, wherein the force applicator further comprises a force transducing mobile plate mounted on the frame for contacting the at least one force sensor, and for one or more of measuring, transmitting and displaying the applied force to qualify and quantify the applied test load into direct translational and/or rotational force.

7. The testing device according to claim 1, wherein the controller is capable of executing a calibration protocol that identifies the specific orientation of each of the first and second position sensors with respect to an anatomical axis of the first and second bone through a performance of predefined motions.

8. The testing device according to claim 7, wherein the calibration protocol includes:
determining a spatial orientation of a proximal segment of the second bone;
determining a spatial orientation of a distal segment of the second bone; and
determining an angular position between the proximal and distal segment about all three axes of rotation in a load bearing condition with the second bone in motion.

9. The testing device according to claim 1, wherein each of the first and second position sensors transmit force and position data signals via a wireless connection to a central processing unit.

10. The testing device according to claim 1, wherein each of the at least one first position sensors are embedded in an elastic stretchable sleeve for surrounding the first bone.

11. The testing device according to claim 1, wherein the at least one first position sensor comprises an array of three or more spatial information sensors spaced apart on the first bone.

12. A method of evaluating the stability of a first joint comprising:
attaching angular position sensors respectively to a first and a second bone above and below the first joint;
attaching at least one force sensor proximate an outer free end of the second bone;
mounting a force applicator proximate an outer free end of the second bone;
applying varus/valgus forces and internal/external rotational torques to the second bone while moving through the flexion-extension range of motion with the first joint;
generating angular motion data with the angular position sensors;
generating force data with the at least one force sensor; and
using a central processing unit in communication with the angular position sensors and the force sensors, for calculating angular positions of the first joint in terms of varus/valgus rotation, flexion/extension and internal/external rotation.

13. The method according to claim 12, further comprising,
with the central processing unit, calculating a laxity envelope for varus/valgus and/or internal/external rotation of the first bone relative to the second bone or vice versa by combining the angular motion data with the force data.

14. The method according to claim 13, further comprising during an activity:
attaching angular position sensors respectively to the first and the second bones above and below the first joint;
attaching at least one force sensor proximate an outer free end of the second bone,
generating angular motion data with the angular position sensors;
generating force data with the at least one force sensor;
implementing post processing algorithms that evaluate mobility of the joint under external stress registered by the force sensors during the activity; and
comparing mobility of the joint during the activity to the laxity envelope.

15. The method according to claim 14, further comprising warning the user if the mobility of the joint exceeds the laxity envelope; wherein the warning comprises an alert on a mobile device generated by an application stored on the mobile device.

16. The method according to claim 14, wherein the step of attaching angular position sensors includes wearing clothing embedded with the angular position sensors covering the first and second bone.

17. The method according to claim 13, wherein the force applicator comprises an anatomically contoured hard shell device that provides sufficient stiffness to a second distal joint to impart reliable force transmission to the first joint across the second bone and the second distal joint.

18. The method according to claim 13, further comprising a calibration protocol that identifies the specific orientation of the individual angular position sensors with respect to an anatomical axis of the first and second bones surrounding the first joint through the performance of predefined motions.

19. The method according to claim 18, wherein the calibration protocol includes:
determining a spatial orientation of a proximal segment of the second bone;
determining a spatial orientation of a distal segment of the second bone; and
determining an angular position between the proximal and distal segment about all three axes of rotation in a load bearing condition with the second bone in motion.

20. A method to use, transfer and apply clinically obtained multi-dimensional laxity data in the field during activities of daily living, rehabilitation and sports activities, whereby the data includes:
in a clinical environment, identifying joint limits comprising angular positions that require elevated or steeply increasing levels of applied forces;
in a non-clinical environment, identifying a set of multi-dimensional joint positions through the range of motion of the joint in relation to external forces required to reach the positions; and
providing direct feedback to the user when said established joint limits are approached or exceeded for a given degree of freedom, where said limits depends on at least one of:
an angular position of the joint;
an angular position of the joint as a function of another angular degree of freedom; and
an angular position of the joint as a function of the clinically required force to reach that position.
